(19) United States
(12) Patent Application Publication
(10) Pub. No.: US 2026/0069296 A1
(43) Pub. Date: Mar. 12, 2026

(54) MULTI-MODALITY BALLOON CATHETER INCLUDING LITHOTRIPSY BALLOON

(71) Applicant: Medtronic, Inc., Minneapolis, MN (US)

(72) Inventors: Ramji Rengarajan, Santa Rosa, CA (US); Jie Wen, Santa Rosa, CA (US)

(21) Appl. No.: 19/105,668

(22) PCT Filed: Aug. 29, 2023
(86) PCT No.: PCT/US2023/031385

§ 371 Date: Feb. 21, 2025

Related U.S. Application Data
(60) Provisional application No. 63/374,032, filed on Aug. 31, 2022.

(51) Int. Cl. A61B 17/22; A61B 17/00; A61M 25/10

(52) U.S. Cl. CPC A61B 17/22022; A61B 17/00234; A61M 25/1011; A61B 2017/00238; A61B 2017/00305; A61B 2017/00477; A61B 2017/00557; A61B 2017/00893; A61B 2017/22025; A61B 2017/22038; A61B 2017/22054; A61B 2017/22062; A61B 2017/22065; A61M 2025/105; A61M 2025/1061; A61M 2025/1084; A61M 2210/12

(57) ABSTRACT

A multi-modality catheter is configured to treat a calcified lesion of a body lumen. The multi-modality catheter includes an integrated intravascular lithotripsy balloon and second treatment balloon. The intravascular lithotripsy balloon includes a shock wave emitter to produce a shock wave for modifying the calcified lesion. The second treatment balloon is configured to treat the calcified lesion.

### FIELD

The present technology is generally related to multi-modality balloon catheter including a lithotripsy balloon, and a method of treating a calcified lesion using the same.

### BACKGROUND

An intravascular lithotripsy (IVL) balloon catheter may be used to modify a calcified lesion within a patient's cardiovascular system. The IVL balloon catheter may have a balloon, such as an angioplasty balloon, at the distal end thereof arranged to be inflated with a fluid. A shock wave is generated in the balloon. The shock wave emitter includes a pair of electrodes, which are coupled to a power source (e.g., high voltage source) at the proximal end of the catheter through a connector. When the inflated balloon is placed adjacent a calcified region of a vein or artery, for example, and a high voltage pulse is applied across the electrodes, a shock wave is formed in the balloon that propagates through the fluid and impinges upon the wall of the balloon and the calcified lesion. Repeated waves modify the calcified lesions. For instance, the shock waves can create weakening or fragmentation of the lesion through the creation of microfractures.

### SUMMARY

The techniques of this disclosure generally relate to a lithotripsy balloon catheter.

In one aspect, the present disclosure is directed to a multi-modality catheter for treating a lesion of a body lumen comprising a catheter body configured to be received in the body lumen. The catheter body has opposite proximal and distal end portions and a longitudinal axis extending between the proximal and distal end portions. An intravascular lithotripsy balloon is coupled to the distal end portion of the catheter body. The intravascular lithotripsy balloon includes i) a balloon body configured to receive fluid to expand the balloon body, and ii) a shock wave emitter received in the balloon and configured to produce a shock wave within the balloon. A second treatment balloon coupled to the distal end portion of the catheter body and spaced apart longitudinally from the intravascular lithotripsy balloon. The second treatment balloon includes a balloon body configured to receive fluid to expand the balloon body. The intravascular lithotripsy balloon and the second treatment balloon are independently expandable.

In another aspect, the disclosure is directed to a method of treating a calcified lesion in a body lumen comprising inserting a multi-modality catheter into the body lumen have the calcified lesion; advancing the multi-modality catheter so that an intravascular lithotripsy balloon of the multi-modality catheter is received in the calcified lesion and at least a portion of a second treatment balloon is disposed outside the calcified lesion, wherein each of the intravascular lithotripsy balloon and the second treatment balloon is unexpanded; delivering fluid into the intravascular lithotripsy balloon to expand the intravascular lithotripsy balloon after said advancing the multi-modality catheter; and generating a shock wave within the intravascular lithotripsy balloon using a shock wave emitter disposed within the intravascular lithotripsy balloon after said delivering fluid into the intravascular lithotripsy balloon to modify the calcified lesion.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a schematic representation of a multi-modality catheter system including a multi-modality catheter, the catheter including an intravascular lithotripsy (IVL) balloon and a second treatment balloon.

FIG. 2 is a perspective of the multi-modality catheter.

FIG. 3 is an enlarged side elevational view of the IVL balloon.

FIG. 4 is an enlarged perspective of a catheter body of the multi-modality catheter shown in cross section.

FIG. 5 is a side elevation of the multi-modality catheter including a first type of second treatment balloon.

FIG. 6 is a side elevation of the multi-modality catheter including a second type of second treatment balloon.

FIG. 7 is a side elevation of the multi-modality catheter including a third type of second treatment balloon.

FIG. 8 is a side elevation of the multi-modality catheter including a fourth type of second treatment balloon.

FIG. 9 is a schematic representation showing the IVL balloon opening a passage in a calcified lesion.

FIG. 10 is a schematic representation showing the second treatment balloon treating the calcified lesion within the opened passage created by the IVL balloon.

FIG. 11 is cross-sectional perspective of another embodiment of a catheter body for the multi-modality catheter.

FIG. 12 is a cross section of the catheter body of FIG. 11.

### DETAILED DESCRIPTION

The treatment of calcified lesions remains a persistent challenge in contemporary interventional practice. For example, calcification is prevalent in chronic total occlusions (CTO), resulting in difficulty with wire and microcatheter crossing. Moreover, calcium alters morphology and compliance of the vessel wall leading to reduced outcome of both angioplasty and stent deployment. Calcification can increase the occurrence of flow limiting dissections and acute vessel recoil, and limit stent efficacy by increasing the risk of incomplete stent expansion, malapposition, and fractures. Additionally, calcified lesion limits drug penetration which significantly reduces the effectiveness of drug delivery treatments such as drug eluting stents and drug coated balloons.

Described herein are multi-modality catheters and methods for treating calcified lesions, whereby a first treatment balloon first deployed and then a second treatment balloon is deployed. In one embodiment, the first balloon is configured as an intravascular lithotripsy (IVL) balloon configured to modify the calcium in the lesion. The IVL balloon is used to gain access through the calcified lesion. Thereafter, the second treatment balloon is used to treat the lesion. As non-limiting examples, the second treatment balloon may include a caged balloon, a drug-coated balloon, a drug-eluting stent, or an injectable porous balloon. The IVL balloon is relatively sized smaller in diameter compared to the second treatment balloon to aid in gaining easier access and entry through the calcified lesions. The IVL balloon may be designed to be a compliant balloon material to reduce potential for vessel injury or dissections and to let the shockwave modify the calcium in the lesion. The second treatment balloon is sized and shaped appropriately to treat lesions of varying sizes (e.g., diameters). Appropriate sizes (diameters and lengths) for the IVL balloon and second treatment balloon can be selected based on lengths and diameters of various diseased arteries and vessels or lesions in the body such as coronary, below-the-knee, common iliac, bile duct, etc. Both the IVL balloon and the second treatment balloon may be designed and constructed to accommodate appropriate French size compatibility with introducer sheath and hemostatic valve at the patient access site. The second treatment balloon may be designed and constructed to have special structures on the shape to have the effect of pillows in order to not cause injury or trauma to the vessel during balloon expansion and full wall apposition for treatment. Further, the second treatment balloon may be designed and constructed to be encapsulated in nitinol structure or a cage to control the balloon expansion and dilatation in order to reduce flow limiting dissections and reduce recoil post procedure.

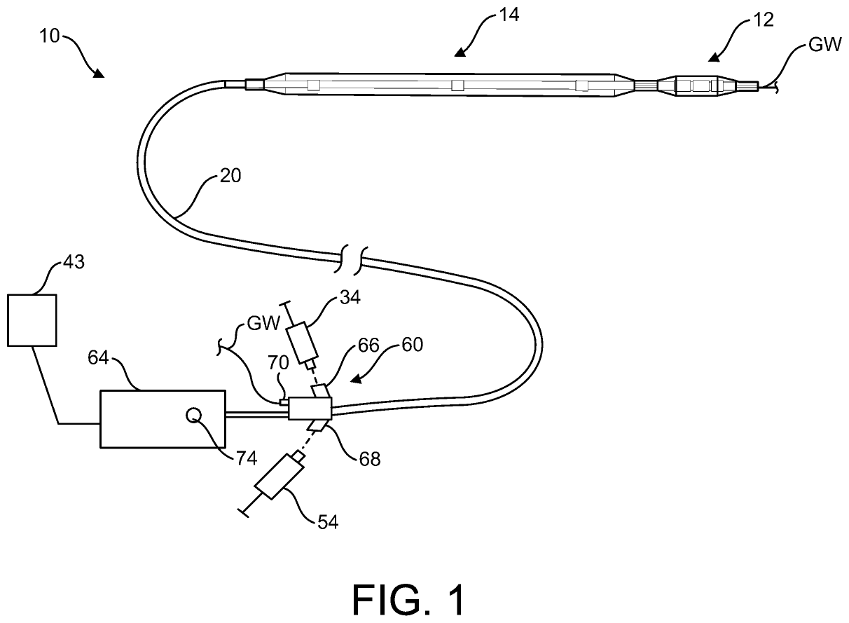

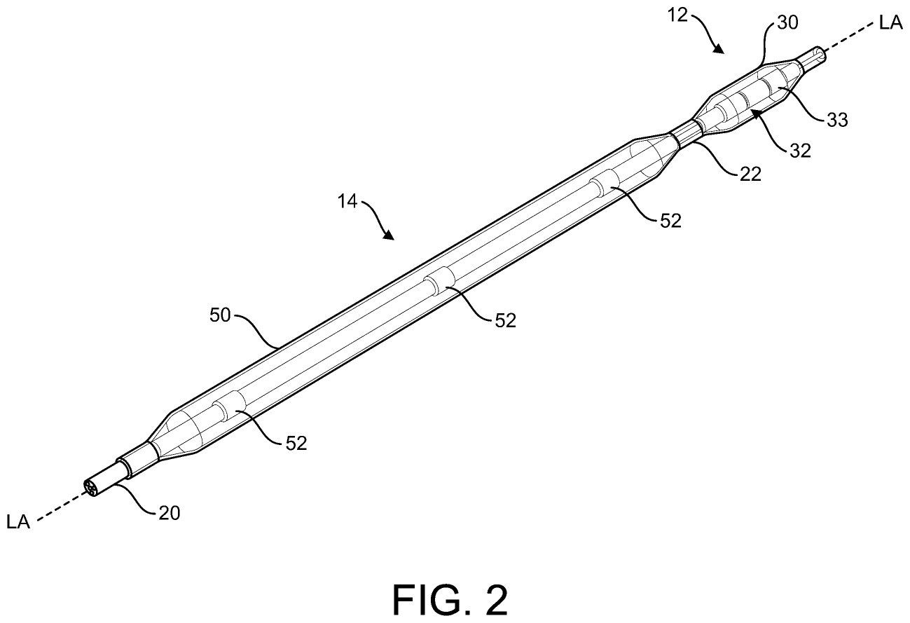

Referring to FIGS. 1 and 2, a multi-modality catheter for treating calcified lesions within the cardiovascular system of a patient is generally indicated at reference numeral 10. The multi-modality catheter 10 generally includes a first treatment balloon, generally indicated at 12), and a second treatment balloon, generally indicated at 14. The first and second treatment balloons 12, 14 are configured to include different treatment modalities. In one example, as illustrated, the first treatment balloon 12 is designed and constructed as an intravascular lithotripsy (IVL) balloon. In the following description, unless referred to separately, the first treatment balloon is referred to as the “IVL balloon.”

The IVL and second treatment balloons 12, 14, respectively, are discrete from one another and integrated into the single multi-modality catheter 10. Thus, the balloons 12, 14 are combined together in the integrated multi-modality catheter 10 so that a single multi-modality catheter functions as both an IVL balloon catheter to modify calcified lesions to gain access to the lesion and a second balloon treatment catheter to further treat the lesion. According to certain embodiments, two or more treatments can be performed without needing to insert and feed separate catheters to the treatment site under fluoroscopy, for example. It is contemplated that the teachings of the present disclosure may be used to treat other body structures, other than blood vessels, for modifying the lesions (e.g., calcified lesions) or other obstructions within the body, such as but not limited to a bile duct, a hepatic duct and a cystic duct.

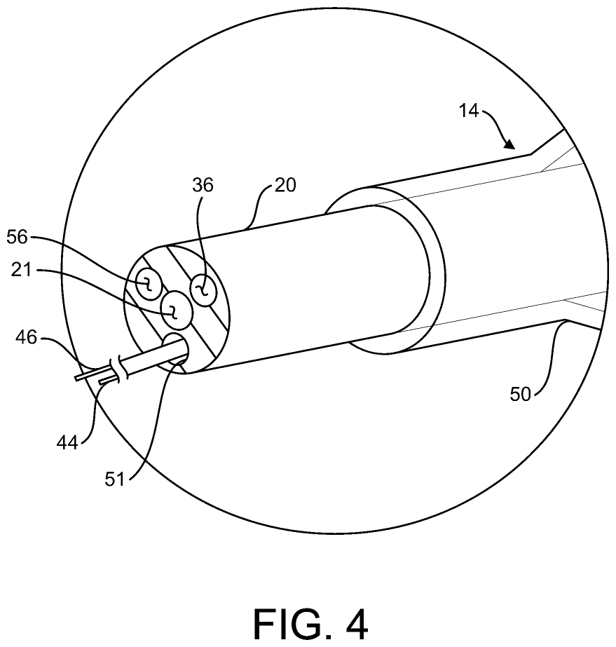

The catheter 10 includes an elongate, flexible catheter body 20 having opposite proximal and distal end portions and a longitudinal axis LA extending therebetween. The body 20 may be formed from a polymer or other material suitable for advancing and tracking the catheter 10 through the patient's vascular system to the target lesion. As explained in more detail below, suitable operating components are coupled or couplable to the proximal end portion of the body 20 for operating the IVL and second treatment balloons 12, 14. As shown in FIG. 4 and explained below, the body 20 defines a plurality of lumens for coupling the operating components to the respective balloons 12, 14. The body 20 also defines a guidewire lumen 21 (FIG. 4) extending through the proximal and distal ends of the body and configured to receive a suitable guidewire (GW) therein. The guidewire lumen 21 may be disposed generally centrally along the body 20.

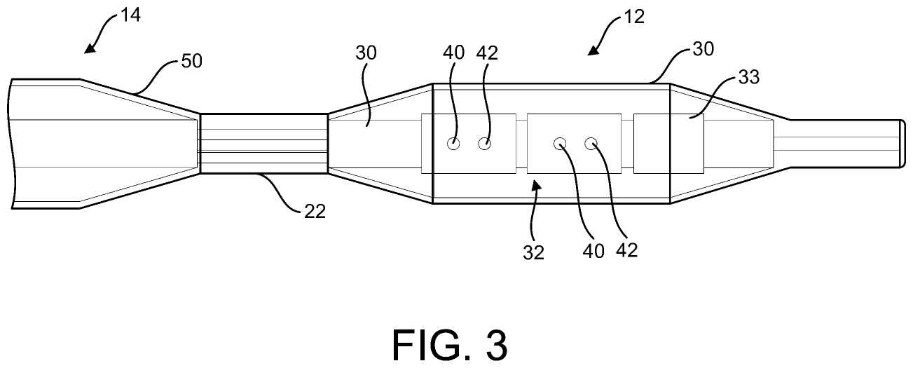

As seen best in FIG. 2, the IVL and second treatment balloons 12, 14 are coupled to the distal end portion of the catheter body 20 and are appropriately spaced apart from one another along the longitudinal axis (LA). In particular, the IVL balloon 12 is distal of the second treatment balloon 14 and generally at the distal end of the catheter body 20. In the illustrated embodiment, as seen best in FIG. 3, a flexible coupler or joint 22 separate the balloons 12, 14 so that the balloons do not overlap or abut one another along the catheter body 20. It is believed the joint 22 allows for the catheter to be more flexibility between the balloons as opposed if the balloons overlapped or abutted one another. However, in one or more embodiments, the joint 22 may be omitted and the balloons 12, 14 may overlap or abut one another. The catheter body 20 may be continuous and extend through the joint 22 between the balloons 12, 14. In one or more other embodiments, the catheter body 20 may be discontinuous including a proximal portion extending through the second treatment balloon 14 to adjacent proximal end of the joint, and a distal portion extending through from adjacent a distal end of the joint and through the IVL balloon 12, whereby the joint 22 provides suitable lumens coupling corresponding lumens of the proximal portion of the body to lumen of the distal portion for the IVL balloon.

As seen best in FIG. 3, the IVL balloon 12 generally includes an expandable balloon body 30 and a shock wave emitter, generally indicated at 32, inside the balloon body. The balloon body 30 may be formed from a suitable polymer material and have suitable compliance and burst pressure, among other parameters, for functioning as an IVL balloon. The balloon body 30 of the IVL balloon 12 may be compliant, semi-compliant, or non-compliant, depending on the desired treatment. One or more markers 33 (e.g., radiopaque material) may be disposed in the balloon body 30, such as on the catheter body 20, to facilitate identification of the balloon 14 under fluoroscopy. The balloon body 30 is coupled to the catheter body 20, such as by welding, adhesion, bonding, overmolding, or in other suitable ways. The balloon body 30 is in fluid communication with a first source of fluid (e.g., saline) 34 (FIG. 1) via a lithotripsy inflation lumen 36 (FIG. 4) extending along the catheter body 20. Fluid is selectively introduced into and removed from the balloon body 30 via the lithotripsy inflation lumen 36 to respectively expand (inflate) and retract (deflate) the IVL balloon 12.

Referring still to FIG. 3, the illustrated shock wave emitter 32 includes at least one pair of first and second electrodes (positive and negative electrodes) or at least one unipolar electrode suitable for producing an electrical arc within fluid of the expanded IVL balloon body 30 when electrical energy is delivered to the electrode(s). In one or more embodiments, the shock wave emitter may be designed and constructed to produce shock waves in other ways other than electrical arcs. For example, a laser or other sources of energy may be used to produce shock waves. The laser or other source of energy may be suitable for producing cavitation and shock waves within the fluid of the balloon 12.

In the illustrated embodiment, the shock wave emitter 32 includes two pairs of first and second electrodes 40, 42, although the shock wave emitter may include more or less electrodes. The electrodes 40, 42 are electrically connected to an electrical power source 43 (e.g., a voltage generator, such as a high voltage generator; FIG. 1) via first and second electrical conductors 44, 46 (e.g., wires or cables; FIG. 4) extending along the catheter body 20 within an electrical conductor lumen 51 of the catheter body 20. In such an embodiment, the electrode pairs may be wired in parallel for simultaneous electrical arcs and shock wave generation. The electrodes can also be individually triggered or triggered in a sequence or a pattern to create a suitable shock wave pattern. The electrical conductors 44, 46 are insulated (e.g., insulated sleeves or coatings) and the electrodes 40, 42 are exposed and uninsulated. In the illustrated embodiment, the first electrodes 40 are electrically connected to a negative terminal of the high voltage (HV) generator 43, and the second electrodes 42 are electrically connected to a positive terminal of the generator.

Voltage pulses from the voltage generator 43 applied to the electrodes 40, 42 produce electrical arcs across the electrodes within the IVL balloon body 30. The electrical arcs in the fluid generate shock waves in the fluid. The shock waves propagate through the fluid and the balloon body 30 to the calcified lesion where the energy can modify the calcified lesions. The voltage generator 43 may be a single voltage, direct current source and may be configured to generate from about 100 to 3000 volts, for example. According to certain embodiments, the system is designed create an electrical arc between electrodes 40, 42. The electrical arc heats the liquid in the balloon to create a vapor bubble. The resulting rapid expansion and contraction from this bubble can create one or more mechanical shock waves, or cavitation bubbles, in the balloon body 30. In certain embodiments, the duration of the shock waves can be as short as on the order of a few microseconds.

The profile (i.e., maximum expanded cross-sectional dimension) of the IVL balloon 12 in one or both of its contracted and expanded states may be less than the profile (i.e., maximum expanded cross-sectional dimension) of the second treatment balloon 14 in one or both of its respective contracted and expanded states. For example, the IVL balloon 12 in its expanded state may have a maximum outer diameter that is less than the outer maximum diameter of the expanded second treatment balloon 14. In addition or alternatively, the IVL balloon 12 in its contracted (e.g., wrapped) state may have a maximum outer diameter that is less than the outer maximum diameter of the contracted (e.g., wrapped) second treatment balloon 14. As an example, the maximum expanded outer diameter of the IVL balloon 12 may be from about 1 mm to about 14 mm. As an example, the maximum contracted outer diameter of the IVL balloon 12 may be from about 0.5 mm to about 4 mm.

As explained below, in an exemplary method lithotripsy is applied to the lesion first using the IVL balloon 12 before treatment using the second treatment balloon 14. Accordingly, the IVL balloon 12 will enter the lesion first and the smaller or slimmer profile facilitates entry of the IVL balloon into the calcified lesion, particularly hard-to-cross lesions, such as totally or substantially occluded lesions. The illustrated IVL balloon 12 also has a length that is less than the length of the second treatment balloon 14. For example, the length of the IVL balloon 12 may be from about 5 mm to about 100 mm. A relatively shorter length facilitates the full length or substantially full length of the IVL balloon 12 being insertable into the lesion for lithotripsy treatment and allows the IVL balloon to be used incrementally as it is advanced through the lesion.

As shown in FIG. 2, the second treatment balloon 14 generally indicated a balloon body 50. The balloon body 50 may be formed from a suitable polymer material and have suitable compliance and burst pressure, among other parameters, for functioning as a second treatment balloon. The balloon body 50 of the second treatment balloon 14 may be compliant, semi-compliant, or non-compliant, depending on the desired treatment. As an example, the maximum expanded outer diameter of the second treatment balloon 14 may be from about 1.5 mm to about 15 mm. As an example, the maximum contracted outer diameter of the second treatment balloon 14 may be from about 0.5 mm to about 4 mm. As an example, the length of the second treatment balloon 14 may be from about 10 mm to about 400 mm. Appropriate size (diameter and balloon lengths) for second treatment balloon can be selected based on diameters and lesions lengths of various diseased arteries and vessels in the body such as coronary, below-the-knee, common iliac, bile duct, etc. The balloon body 50 is coupled to the catheter body 20, such as by welding, adhesion, bonding, overmolding, or in other suitable ways. One or more markers 52 (e.g., radiopaque material) may be disposed in the balloon body 50, such as on the catheter body 20, to facilitate identification of the balloon 14 under fluoroscopy. The balloon body 50 is in fluid communication with a source of fluid (e.g., saline) 54 via a treatment inflation lumen 56 extending along the catheter body 20. Fluid is selectively introduced into and removed from the balloon body 50 via the IVL inflation lumen 36 to respectively expand (inflate) and retract (deflate) the second treatment balloon 14.

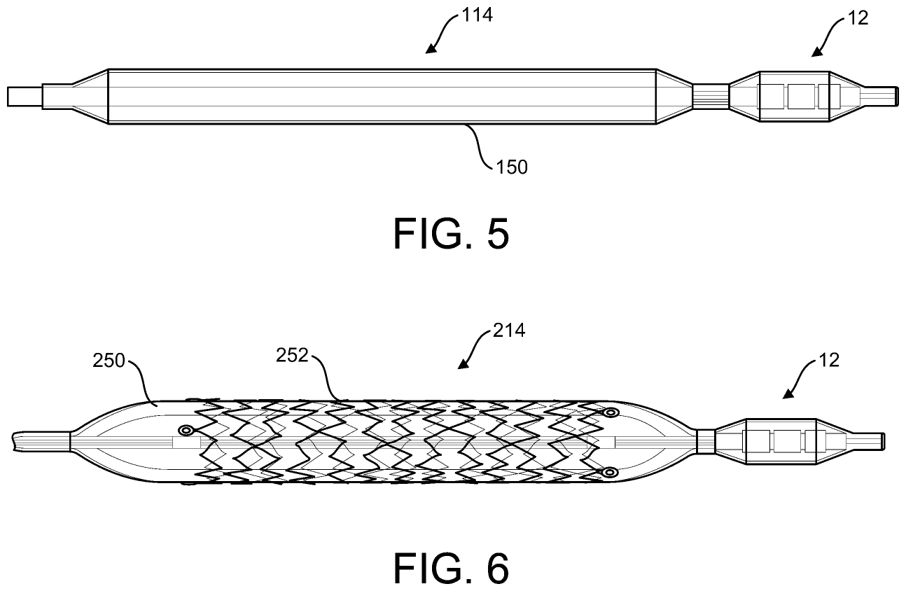

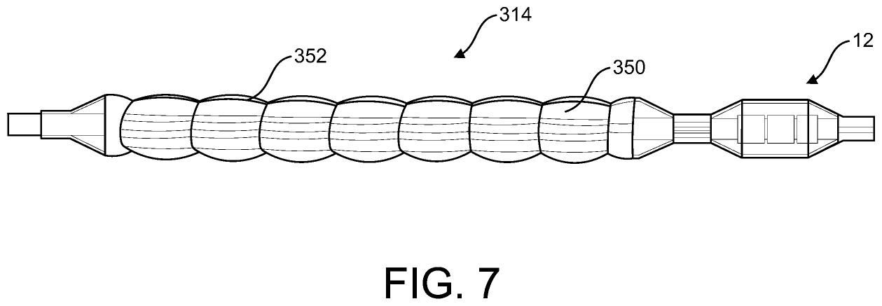

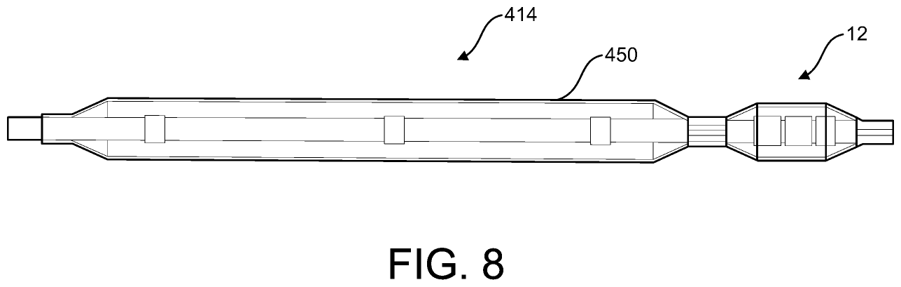

In general, the second treatment balloon 14 may be designed and constructed as a suitable cardiovascular treatment balloon for treating the lesion after lithotripsy using the IVL balloon 12. Among other non-limiting examples, the second treatment balloon may be designed and constructed as: i) a drug-coated balloon (DCB) 114 shown in FIG. 5, where the balloon body 150 is coated with a therapeutic agent for treating the lesion; ii) a drug-eluting stent balloon 214 shown in FIG. 6, which includes a drug-eluting stent 252 disposed on and to be deployed by the balloon body 250; iii) a caged balloon 314 (e.g., nitinol-cage) shown in FIG. 7, where a cage 352 surrounds the balloon body 350 to control dilation (e.g., the CHOCOLATE PTA balloon sold by Medtronic PLC); or iv) drug-injectable balloon 414 shown in FIG. 8, where the balloon body 450 is porous to enable paclitaxel or another therapeutic agent to be delivered to the lesion through balloon body. The therapeutic agent can include anti-proliferative, anti-inflammatory, antineoplastic, antiplatelet, anticoagulant, anti-fibrin, antithrombotic, antimitotic, antibiotic, antiallergic and antioxidant compounds. Suitable therapeutic agent may include antiproliferative or anti-inflammatory agents such as paclitaxel, sirolimus (rapamycin) and their chemical derivatives or analogues which are mTOR inhibitors, inhibitory RNA, inhibitory DNA, steroids and complement inhibitors. In some embodiments, the therapeutic agent is selected from the group consisting of zotarolimus, everolimus, sirolimus, deforolimus, tacrolimus, temsirolimus, pimecrolimus, novolimus, myolimus, paclitaxel, protaxel, and derivatives and combinations thereof. Additionally, excipients can be added to the drug formulation to aid drug dispersion, transfer, and adsorption. The excipient is selected from the group consisting of urea, magnesium stearate, acetyl tributyl citrate, carboxymethyl cellulose, sodium carboxymethyl cellulose, diethanolamine carboxymethyl cellulose, carboxymethyl cellulose derivatives, polysorbates, TWEEN™ 20 (polysorbate 20), TWEEN™ 80 (polysorbate 80), poly(vinyl alcohol), lecithin, gelatin, sucrose, 1,2-distearoyl-sn-glycero-3-phospho-ethanolamine-N-[methoxy(polyethylene glycol)-2000](ammonium salt) (PEG-PE), phosphatidyl choline, phospholipids, pegylated phospholipids, TWEEN™ 60 (polysorbate 60), vitamin E TPGS, PLURONIC® 68 which is a poly(ethylene oxide)-poly(propylene oxide) block copolymers, poly(ethylene oxide)-poly(propylene oxide) block copolymers, poloxamers 188 and 407, ascorbyl palmitate, CREMOPHOR EL™, fatty alcohols, ammonium salts, fatty esters, tocopherols, phospholipids and combinations thereof.

Referring to FIG. 1, in the illustrated embodiment, a coupler, generally indicated at 60, is connected to a proximal end portion of the catheter body 20. The coupler 60 is configured to couple a control handle 64 to the catheter 10. In the illustrated embodiment, the coupler 60 is configured to electrically and mechanically couple the control handle 64 to the catheter body 20. The illustrated coupler 60 includes a first port 66 configured to couple the source of fluid 34 (e.g., saline in a syringe) to the IVL inflation lumen 36 of the catheter body 20 for selectively expanding the IVL balloon 12; a second port 68 configured to couple the source of fluid 54 (e.g., saline in a syringe) to the treatment inflation lumen 56 of the catheter body for selectively expanding the second treatment balloon 14; and a third port 70 in communication with the guidewire lumen 21 of the catheter body and configured to receive the guidewire (GW) in the guidewire lumen. It is understood that the coupler 30 may be of other configurations and/or designs or the coupler may be omitted and replaced with other components for accomplishing one or more of the functions of the coupler. The control handle 64 is electrically connected to the source of electrical power 43 and is configured to enable an operator to selectively deliver the electrical power to the shock wave emitter 32. The illustrated control handle 64 includes an actuator 74 (e.g., a button) for controlling the activation of the IVL power. The control handle may have other features and may include a different user interface. Together, the multi-modality catheter 10, the coupler 60, the control handle 64, and the electrical power source 43 make up a multi-modal catheter system.

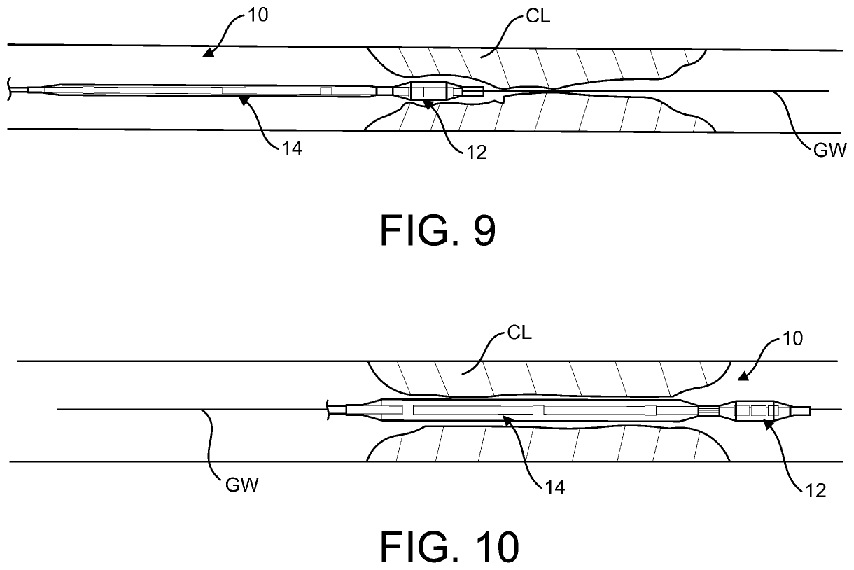

Referring to FIGS. 9 and 10, in an exemplary method of use, the multi-modality catheter 10 is used to open an occluded or partially occluded lumen, such as a blood vessel. The catheter 10 is feed along the guidewire (GW) through a blood vessel to a calcified lesion (CL), such as by using fluoroscopy. The IVL balloon 12, with its relatively slim profile, is first inserted into the calcified lesion. As shown, the second treatment balloon 14 may be proximal of the CL, at least when the IVL balloon is first inserted into the lesion. With the IVL balloon at least partially within the CL, the balloon body 30 is inflated with fluid from the fluid source 34 and then electrical energy is supplied to the shock wave emitter to emit shock waves from the balloon to modify the calcified lesions and widen the passage through the lesion. The second treatment balloon 14 may remain uninflated. The balloon 12 is then deflated and can be advanced distally within the CL to continue widening the passage through the lesion in the distal direction. The process can continue until the passage is widened through the proximal and distal ends of the lesion. After widening the passage with the IVL balloon 12, the second treatment balloon 14 is employed to treat the CL without needing to remove the catheter from the body lumen. The second treatment balloon 14 is positioned in the CL and expanded by introducing fluid from the fluid source 54 into the balloon body 50. In another example, the IVL balloon 12 and the second treatment balloon 14 may be used simultaneously to treat the lesion during at least one step of the treatment procedure. After suitable treatment, and with each of the balloons 12, 14 deflated, the catheter 10 is withdrawn from the body lumen.

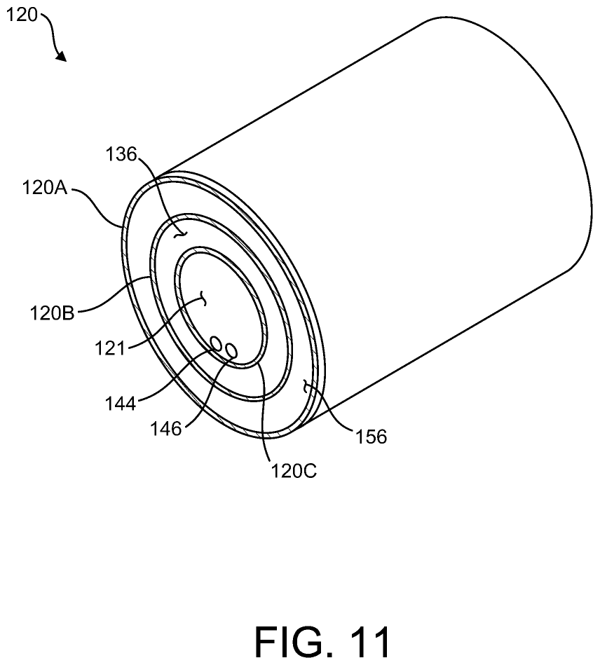

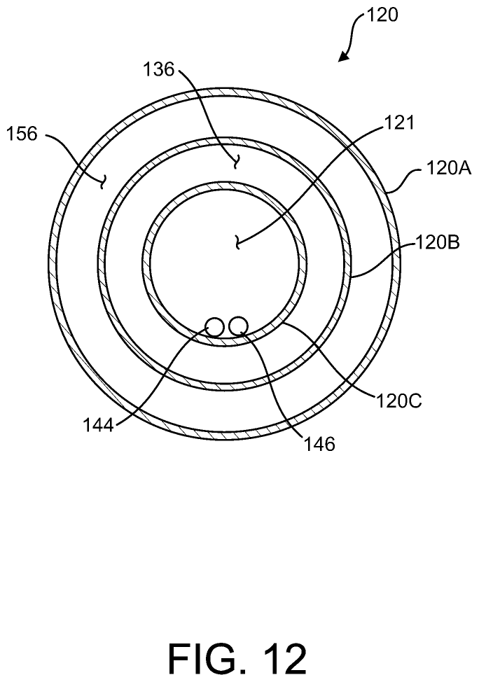

Referring to FIGS. 11 and 12, another embodiment of a catheter body for use with the catheter and catheter system set forth above herein is generally indicated at reference numeral 120. This catheter body 120 is suitable for incorporation in the catheter system 10 described above by substituting it for the catheter body 20. The catheter body 120 includes first, second, and third coaxial layers 120A, 120B, 120C, respectively, defining coaxial a guidewire lumen 121 for receiving the guidewire GW, an IVL inflation lumen 136 configured to be in fluid communication with the IVL balloon for inflation thereof, and a treatment inflation lumen 156 configured to be in fluid communication with the second treatment balloon for inflation thereof. Each of the layers 120A, 120B, 120C and the lumens 121, 136, 156 extend along the length of the catheter body 120. In the illustrated embodiment the first layer 120A is a radially inner layer and defines the guidewire lumen 121. The second layer 120B is a radially intermediate layer disposed radially between the first and third layers 120A, 120C. The second layer 120B is spaced radially apart from the first layer 120A. Together, the first layer 120A and the second layer 120B define the IVL inflation lumen 136. The third layer 120C is a radially outer layer and is radially spaced apart from the second layer 120B. Together, the second layer 120B and the third layer 120C define the treatment inflation lumen 156. In the illustrated embodiment, first and second electrical conductors 144, 146 (e.g., wires or cables) extend along the catheter body 120 within the guidewire lumen 121, one or more of the other lumens, 136, 156, or one or more of the layers 120A, 120B, 120C. The electrical conductors 144, 146 may be identical in structure and function as the electrical conductors 44, 46 described above.

Each of the layers 120A, 120B, 120C may be flexible and comprise (e.g., formed from) a suitable, fluid impermeable material, such as a suitable polymer and blends thereof. The first layer 120A may be lubricious and/or formed from material (e.g., polymer) having a low coefficient of friction. In an exemplary embodiment, the first layer 120A comprises a polytetrafluoroethylene (PTFE), including blends thereof, but other materials can be used in one or more embodiments. The inner surface of the inner layer 120A can also be coated with a lubricious coating in one or more embodiments. The third layer 120C can comprise the same material as the first layer 120A or a different material. For example, the third layer 120C may comprise a polyether block amide (i.e., a PEBA), such as PEBAX 55D commercially available from Arkema. Forming the third layer 120C from a PEBA is believed to enhance the radial crush resistance of the catheter body 120. Other materials can be used for the outer layer in one or more embodiments. The second layer 120B may also comprise a polymer, which may be different from the polymers of the first and third layers 120A, 120C. In one embodiment, the second layer 120B may comprise a polyimide, including blends thereof. The second layer 120B may include a reinforcing coil, braid, fibers, or other structure(s) for increasing radial strength of the layer to inhibit collapse and over-expansion of the IVL and treatment inflation lumens 136, 156, respectively.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

The invention may be further described by reference to the following numbered paragraphs:
1. A multi-modality catheter for treating a lesion of a body lumen comprising:
   a catheter body configured to be received in the body lumen, the catheter body having opposite proximal and distal end portions and a longitudinal axis extending between the proximal and distal end portions;
   an intravascular lithotripsy balloon coupled to the distal end portion of the catheter body, wherein the intravascular lithotripsy balloon includes i) a balloon body configured to receive fluid to expand the balloon body, and ii) a shock wave emitter received in the balloon and configured to produce a shock wave within the balloon; and
   a second treatment balloon coupled to the distal end portion of the catheter body and being spaced apart longitudinally from the intravascular lithotripsy balloon, wherein the second treatment balloon includes a balloon body configured to receive fluid to expand the balloon body,
   wherein the intravascular lithotripsy balloon and the second treatment balloon are independently expandable.
2. The multi-modality catheter set forth in paragraph 1, wherein the balloon body of the intravascular lithotripsy balloon has a maximum cross-sectional dimension that is less than a maximum cross-sectional dimension of the balloon body of the second treatment balloon.
3. The multi-modality catheter set forth in paragraph 2, wherein the balloon body of the intravascular lithotripsy balloon has a length that is less than a length of the balloon body of the second treatment balloon.
4. The multi-modality catheter set forth in paragraph 1, wherein the catheter body includes a flexible joint coupling the second treatment balloon to the intravascular lithotripsy balloon.
5. The multi-modality catheter set forth in paragraph 1, wherein the catheter body defines a lithotripsy inflation lumen extending along the longitudinal axis in fluid communication with the intravascular lithotripsy balloon, and a separate treatment inflation lumen extending along the longitudinal axis in fluid communication with the second treatment balloon.
6. The multi-modality catheter set forth in paragraph 5, further comprising at least one electrical conductor coupled to the shock wave emitter, wherein the at least one electrical conductor is received in an electrical conductor lumen extending along the longitudinal axis of the catheter body.
7. The multi-modality catheter set forth in paragraph 1, wherein the catheter body defines a guidewire lumen extending along the longitudinal axis configured to receive a guidewire therein.
8. The multi-modality catheter set forth in paragraph 1, wherein the shock wave emitter includes at least one electrode configured to produce an electrical arc when electrical energy is applied to the at least one electrode thereby creating a shock wave within the balloon.
9. The multi-modality catheter set forth in paragraph 8, in combination with a source of electrical energy electrically coupled to the at least one electrode.
10. The multi-modality catheter set forth in paragraph 1, wherein the second treatment balloon is a drug-coated balloon.
11. The multi-modality catheter set forth in paragraph 1, wherein the second treatment balloon is a drug-eluting stent balloon.
12. The multi-modality catheter set forth in paragraph 1, wherein the second treatment balloon is a caged balloon.
13. The multi-modality catheter set forth in paragraph 1, wherein the second treatment balloon is a drug-injectable balloon.
14. The multi-modality catheter set forth in paragraph 1, wherein the intravascular lithotripsy balloon is distal of the second treatment balloon on the catheter body.
15. A method of treating a calcified lesion in a body lumen comprising:
   inserting a multi-modality catheter into the body lumen having the calcified lesion;
   advancing the multi-modality catheter so that an intravascular lithotripsy balloon of the multi-modality catheter is received in the calcified lesion and at least a portion of a second treatment balloon is disposed outside the calcified lesion, wherein each of the intravascular lithotripsy balloon and the second treatment balloon is unexpanded;
   delivering fluid into the intravascular lithotripsy balloon to expand the intravascular lithotripsy balloon after said advancing the multi-modality catheter; and
   generating a shock wave within the intravascular lithotripsy balloon using a shock wave emitter disposed within the intravascular lithotripsy balloon after said delivering fluid into the intravascular lithotripsy balloon to modify the calcified lesion.
16. The method of treating the calcified lesion in the body lumen set forth in paragraph 15, wherein the second treatment balloon remains uninflated during said generating a shock wave within the intravascular lithotripsy balloon.
17. The method of treating the calcified lesion in the body lumen set forth in paragraph 15, further comprising:
   further advancing, after said generating a shock wave, the multi-modality catheter so that the uninflated second treatment balloon of the multi-modality catheter is received in the lesion that needs treatment; and
   delivering fluid into the second treatment balloon to expand the second treatment balloon after said further advancing the multi-modality catheter.
18. The method of treating the calcified lesion in the body lumen set forth in paragraph 17,
wherein said delivering fluid into the intravascular lithotripsy balloon comprises delivering fluid into a lithotripsy inflation lumen defined by a catheter body of the multi-modality catheter, the lithotripsy inflation lumen being in fluid communication with the intravascular lithotripsy balloon, wherein said delivering fluid into the second treatment balloon comprises delivering fluid into a separate treatment inflation lumen defined by the catheter body of the multi-modality catheter, the treatment inflation lumen being in fluid communication with the second treatment inflation balloon.
19. The method of treating the calcified lesion in the body lumen set forth in paragraph 15, wherein a balloon body of the intravascular lithotripsy balloon has a maximum cross-sectional dimension that is less than a maximum cross-sectional dimension of a balloon body of the second treatment balloon.
20. The method of treating the calcified lesion in the body lumen set forth in paragraph 19, wherein the balloon body of the intravascular lithotripsy balloon has a length that is less than a length of the balloon body of the second treatment balloon.

1-15. (canceled)

16: A multi-modality catheter for treating a lesion of a body lumen comprising:
a catheter body configured to be received in the body lumen, the catheter body having opposite proximal and distal end portions and a longitudinal axis extending between the proximal and distal end portions;
an intravascular lithotripsy balloon coupled to the distal end portion of the catheter body, wherein the intravascular lithotripsy balloon includes i) a balloon body configured to receive fluid to expand the balloon body, and ii) a shock wave emitter received in the balloon and configured to produce a shock wave within the balloon; and
a second treatment balloon coupled to the distal end portion of the catheter body and being spaced apart longitudinally from the intravascular lithotripsy balloon, wherein the second treatment balloon includes a balloon body configured to receive fluid to expand the balloon body,
wherein the intravascular lithotripsy balloon and the second treatment balloon are independently expandable.

17: The multi-modality catheter set forth in claim 16, wherein the balloon body of the intravascular lithotripsy balloon has a maximum cross-sectional dimension that is less than a maximum cross-sectional dimension of the balloon body of the second treatment balloon.

18: The multi-modality catheter set forth in claim 17, wherein the balloon body of the intravascular lithotripsy balloon has a length that is less than a length of the balloon body of the second treatment balloon.

19: The multi-modality catheter set forth in claim 16, wherein the catheter body includes a flexible joint coupling the second treatment balloon to the intravascular lithotripsy balloon.

20: The multi-modality catheter set forth in claim 16, wherein the catheter body defines a lithotripsy inflation lumen extending along the longitudinal axis in fluid communication with the intravascular lithotripsy balloon, and a separate treatment inflation lumen extending along the longitudinal axis in fluid communication with the second treatment balloon.

21: The multi-modality catheter set forth in claim 20, further comprising at least one electrical conductor coupled to the shock wave emitter, wherein the at least one electrical conductor is received in an electrical conductor lumen extending along the longitudinal axis of the catheter body.

22: The multi-modality catheter set forth in claim 16, wherein the catheter body defines a guidewire lumen extending along the longitudinal axis configured to receive a guidewire therein.

23: The multi-modality catheter set forth in claim 16, wherein the shock wave emitter includes at least one electrode configured to produce an electrical arc when electrical energy is applied to the at least one electrode thereby creating a shock wave within the balloon.

24: The multi-modality catheter set forth in claim 23, in combination with a source of electrical energy electrically coupled to the at least one electrode.

25: The multi-modality catheter set forth in claim 16, wherein the second treatment balloon is a drug-coated balloon.

26: The multi-modality catheter set forth in claim 16, wherein the second treatment balloon is a drug-eluting stent balloon.

27: The multi-modality catheter set forth in claim 16, wherein the second treatment balloon is a caged balloon.

28: The multi-modality catheter set forth in claim 16, wherein the second treatment balloon is a drug-injectable balloon.

29: The multi-modality catheter set forth in claim 16, wherein the intravascular lithotripsy balloon is distal of the second treatment balloon on the catheter body.

30: A method of treating a calcified lesion in a body lumen comprising:
inserting a multi-modality catheter into the body lumen have the calcified lesion;
advancing the multi-modality catheter so that an intravascular lithotripsy balloon of the multi-modality catheter is received in the calcified lesion and at least a portion of a second treatment balloon is disposed outside the calcified lesion, wherein each of the intravascular lithotripsy balloon and the second treatment balloon is unexpanded;
delivering fluid into the intravascular lithotripsy balloon to expand the intravascular lithotripsy balloon after said advancing the multi-modality catheter; and
generating a shock wave within the intravascular lithotripsy balloon using a shock wave emitter disposed within the intravascular lithotripsy balloon after said delivering fluid into the intravascular lithotripsy balloon to modify the calcified lesion.

31: The method of treating the calcified lesion in the body lumen set forth in claim 30, wherein the second treatment balloon remains uninflated during said generating a shock wave within the intravascular lithotripsy balloon.

32: The method of treating the calcified lesion in the body lumen set forth in claim 30, further comprising:
further advancing, after said generating a shock wave, the multi-modality catheter so that the uninflated second treatment balloon of the multi-modality catheter is received in the lesion that needs treatment; and
delivering fluid into the second treatment balloon to expand the second treatment balloon after said further advancing the multi-modality catheter.

33: The method of treating the calcified lesion in the body lumen set forth in claim 32,
wherein said delivering fluid into the intravascular lithotripsy balloon comprises delivering fluid into a lithotripsy inflation lumen defined by a catheter body of the multi-modality catheter, the lithotripsy inflation lumen being in fluid communication with the intravascular lithotripsy balloon,
wherein said delivering fluid into the second treatment balloon comprises delivering fluid into a separate treatment inflation lumen defined by the catheter body of the multi-modality catheter, the treatment inflation lumen being in fluid communication with the second treatment inflation balloon.

34: The method of treating the calcified lesion in the body lumen set forth in claim 30, wherein a balloon body of the intravascular lithotripsy balloon has a maximum cross-sectional dimension that is less than a maximum cross-sectional dimension of a balloon body of the second treatment balloon.

35: The method of treating the calcified lesion in the body lumen set forth in claim 34, wherein the balloon body of the intravascular lithotripsy balloon has a length that is less than a length of the balloon body of the second treatment balloon.
